SEQUENCE LISTING (3 sequences)

SEQ ID NO: 1 - protein, 29 aa, Artificial Sequence
  Synthesized Peptide
NWGRVVALFYFASKLVLKALSTKVPELIR

SEQ ID NO: 2 - protein, 14 aa, Artificial Sequence
  Synthesized Peptide
KLAKLAKKLAKLAK

SEQ ID NO: 3 - protein, 8 aa, Artificial sequence
  Synthesized Peptide
RRRRRRRR

(19) United States
(12) Patent Application Publication
(10) Pub. No.: US 2012/0289472 A1
(43) Pub. Date: Nov. 15, 2012

(54) MOLECULES CAPABLE OF INDUCING CELL DEATH BY TARGETING THE MITOCHONDRIA AND APPLICATIONS THEREOF

(76) Inventor: Abdel Aouacheria, Saint-Etienne (FR)

(21) Appl. No.: 13/394,941

(22) PCT Filed: Sep. 8, 2010
(86) PCT No.: PCT/IB2010/054052

§ 371 Date: Mar. 8, 2012

(30) Foreign Application Priority Data
Sep. 9, 2009 (FR) 09/04301

(51) Int. Cl. C07K 14/00; A61K 38/16; A61K 8/64; C12N 5/071

(52) U.S. Cl. 514/21.3; 435/375; 530/324

(57) ABSTRACT

The invention relates to the use of derivatives of the membrane insertion regions of proteins of the Bcl-2 family to induce cell death by targeting the mitochondria, characterised in that said derivatives are peptides belonging to regions α5 and/or α6 of the Bax protein or equivalent regions found in the other proteins of the Bcl-2 family.

The subject of the invention is the use of isolated peptides for inducing cell death by targeting the mitochondrion, and biological applications thereof.

In addition to providing the energy essential to cell survival, the mitochondrion performs essential functions in the control of apoptosis in vertebrates.

The mitochondrial apoptosis pathway is defined by a major event: permeabilization of the outer mitochondrial membrane, which results in the release into the cytoplasm of toxic proteins (such as, for example, cytochrome c) that are normally contained in the intermembrane space.

The integrity of this membrane is under the control of the proteins of the Bcl-2 family. The proteins of this family, which are either pro-apoptotic (such as Bax and Bid) or anti-apoptotic (such as Bcl-2 and Bcl-xL), form homodimers and heterodimers, the whole of these protein-protein interactions participating in the regulation of apoptosis.

However, it is known that interaction of the proteins of the Bcl-2 family with intracellular membranes is essential for their function. Thus, both the pro-apoptotic proteins (such as Bax and Bid) and the anti-apoptotic proteins (such as Bcl-2 and Bcl-xL) of the Bcl-2 family can form pores in vitro in artificial membranes. Experiments also indicate that composite channels formed in the outer mitochondrial membrane by oligomers of the pro-apoptotic proteins Bax and Bak (alone or in combination with other proteins) allow solutes such as cytochrome c to pass through.

Proteins homologous to Bcl-2 share a globular structure (resembling that of certain bacterial toxins which form membrane pores) comprising between 6 and 9 amphipathic alpha-helices. Several structural studies have shown that one of the essential structural determinants for the function of these proteins is a supersecondary (“helix-turn-helix”) motif composed of two anti-parallel “hairpin” alpha-helices (alpha5-alpha6 in Bax), capable of being inserted into biological membranes and of forming pores. The 3D structure of Bax is given in FIG. 1. Each helix of the motif has a size of approximately 15-20 amino acids (order of magnitude for an alpha-helix capable of crossing a lipid bilayer), separated by a turn made up of 3-4 residues. While the available studies support the importance of this domain for the insertion of Bcl-2 family proteins into artificial membranes and the formation of pores in model membrane systems (for review, see Petros et al., Biochim. Biophys. Acta, 2004), they do not provide information on the mechanism of action of this motif at the mitochondrial level. Thus, the exact contribution of this motif to pore formation in cellulo and in vivo by the proteins of the Bcl-2 family is still largely speculative.

It has been reported in the literature that peptides corresponding to the central membrane insertion helices (alpha5 and alpha6) of Bax are capable of inducing, by themselves, the release of molecules contained in liposomes (Garcia-Saez et al., Biophys J, 2005; Garcia-Saez et al., FEBS J, 2006; Garcia-Saez et al., Biophys J, 2007). Furthermore, a recent structural study has shown that a peptide corresponding to the alpha5 helix of Bax forms a lipid pore (Qian et al. Proc Natl Acad Sci USA, 2008). Although these studies cast light on the fundamental mechanisms of pore formation in artificial and acellular systems, no indication is found regarding the ability, of the peptides used, to induce cell death, and their effect on mitochondria has not been explored.

One study (Ishibashi et al. Biochem Biophys Res Commun, 1998) has indicated that ectopic expression, by bacteria or by mammalian cells, of constructs encoding Bax fragments comprising the alpha5 and/or alpha6 helices exerts a toxic effect. The authors of the study report that the Bax regions capable of killing mammalian cells are the same as those that induce the death of bacteria, which are devoid of mitochondria. On the basis of this study, it is not possible to determine the mechanism of action of the fragments expressed, and in particular to know whether the cytotoxic activity observed with the mammalian cells depends on an action at the mitochondrial level. Notably, the study makes reference only to the use of recombinant plasmids, the effect of peptides corresponding to the alpha5 and/or alpha6 regions of Bax not having been tested.

It is likely that the membrane insertion regions of the proteins of the Bcl-2 family have been selected by evolution so as to specifically integrate into the mitochondrial membranes of animal cells. It is important to experimentally test whether these regions, outside the overall context of the proteins that contain them, are capable of affecting the mitochondria, of interfering with their structure (such as, for example, their membranes) and/or their function (such as, for example, their transmembrane potential) and of causing cell death. At the current time, the capacity of peptides deriving from this membrane insertion motif to trigger an apoptotic process by themselves by acting on the mitochondrion is not supported by any experimental evidence. Such peptides could represent an alternative to the artificial cationic peptides used to trigger cell death (Ellerby et al. Nat Med, 1999), which are not very active and therefore not very suitable for industrial development.

The work by the inventors has related to the study of peptides derived from these regions in order to determine whether they can by themselves target mitochondrial membranes and cause cell death by apoptosis.

The results obtained have made it possible to identify minimum structural determinants that direct the interaction of the proteins of the Bcl-2 family with the membranes. Advantageously, some regions have proven to be of great interest for targeting the mitochondrion and destabilizing mitochondrial membranes. On the basis of these regions, bioactive peptides have been developed which open up a route of access to new molecules that can be used in therapy.

These new molecules have been denoted “poropeptides” (from the Greek word Poros ΠOPOΣ: hole or maritime passage; figuratively: effective means for circumventing a difficulty), a poropeptide denoting a peptide which has the ability to form pores in lipid membranes or to destabilize biological membranes such as mitochondrial membranes.

Poropeptides, designed on the basis of the membrane insertion regions of the proteins of the Bcl-2 family, have thus proven to be capable of inducing the apoptosis of harmful cells, such as, for example, tumor cells.

The objective of the invention is therefore the use of peptides of which the sequence derives from the membrane insertion regions of the proteins of the Bcl-2 family for inducing cell death by targeting the mitochondrion.

The invention is also directed toward providing new molecules of peptides developed from these regions.

The invention is also directed toward providing, with these various molecules, highly effective means for restoring an apoptosis process in cancer cells while at the same time limiting as much as possible the repercussions on the surrounding healthy cells. This targeting step may, for example, be based on vectorization of the peptides by coupling to a homing molecule which provides selective targeting thereof at the level of the tumor site, whilst sparing the healthy tissues. Since all the living cells of the organism (healthy or pathological) have mitochondria, the poropeptides that induce rupturing of mitochondrial permeability can be used to induce the death of numerous cell types. With the progress in therapeutic targeting techniques, it may be envisioned to carry out the selective elimination of certain cell populations harmful to the organism.

The invention thus relates to the use of derivatives of the membrane insertion helices of the proteins of the Bcl-2 family for inducing cell death by targeting the mitochondrion, characterized in that the derivatives are peptides corresponding to the α5 and/or α6 regions of the Bax protein or to the equivalent regions present in the other proteins of the Bcl-2 family.

The invention also relates to derivatives, variants, mutants or fragments of the abovementioned peptides.

Synthetic peptides derived from those defined above comprise, for example, means for cell penetration or recognition of a particular cell type.

Advantageous means correspond to a transduction motif which allows their internalization into the targeted cell. It is in particular a polyarginine motif, advantageously comprising 8 arginine residues. Other modifications correspond to deletions and/or substitutions of one or more amino acids. Thus, one or more cysteine residues of said regions can be replaced with serine or glycine motifs.

The invention also relates to conjugates which contain a peptide derived from those defined above and a targeting molecule, such as molecules which recognize the endothelium, making it possible to target a particular tissue or cell type, such as certain tumor cells or angiogenic endothelial cells.

The invention also relates to functional equivalents of the peptides defined above, such as peptides comprising modifications resulting from post-translational processes, for instance glycosylation, or from chemical modifications such as coupling with lipids, sugars, nucleotide sequences or amino acid sequences, provided that these modifications do not modify the pro-apoptotic activity of said peptides in accordance with the tests given in the experimental section hereinafter. The functional equivalents also comprise peptides of which one or more amino acids are D-conformation amino acids. The invention also covers the retro peptides and the retro-inverso peptides.

The invention is directed in particular toward the peptides derived from the α5 and α6 regions, by way of example, the peptide of sequence SEQ ID No. 1: NH₂-NWGRVVALFYFASKLVLKALSTKVPELIR-COOH.

After internalization, these “mitochondriotoxic” compounds are capable of triggering apoptosis by destabilizing the mitochondrial membranes, in particular the outer mitochondrial membrane. Advantageously, they induce the release of multiple toxic molecules contained in the intermembrane space of the mitochondria, in particular cytochrome c.

As illustrated by the examples, their cytotoxic activity has been demonstrated in cellulo.

Thus, according to another aspect, the invention is directed toward the use of the peptides defined above, as medicaments.

The pharmaceutical compositions of the invention are characterized in that they comprise a therapeutically effective amount of at least one peptide as defined above, in combination with a pharmaceutically acceptable vehicle.

The present invention also relates to a pharmaceutical composition comprising, as active ingredient, at least one peptide as defined previously, combined in said composition with one or more pharmaceutically acceptable vehicles, diluents or excipients.

The administration of a pharmaceutical composition according to the invention can be carried out according to the techniques known to those skilled in the art by any one of the methods of administration accepted for therapeutic agents. These processes comprise the systemic, topical, oral or else central administration, for example via surgery or else via intraocular administration. The subcutaneous implantation of biodegradable implants may also be mentioned.

The dosage for the administration of the peptides according to the invention is selected according to many factors, including the type, the species, the age, the weight, the sex and the medical condition of the individual; the seriousness of the condition to be treated; the route of administration; the condition of the individual's kidney and liver functions and the nature of the particular compound, or salt, used. The normally experienced person skilled in the art will determine and will prescribe the effective amount for preventing, impeding or stopping the progress of the medical condition to be treated.

By virtue of their direct effect on mitochondrial permeability, the above compounds are particularly advantageous for triggering an apoptotic process after internalization into tumor cells or cells of neovessels irrigating tumors.

Owing to their ability to target mitochondria, a subject of the invention is also the use of the above-mentioned peptides as mitochondrial targeting molecules for delivering a compound or a polypeptide to mitochondrial membranes in order to induce apoptosis.

They can generally be used in situations with an increase in neovascularization, such as rheumatoid arthritis or obesity. This is because sustained angiogenesis characterizes the severe forms of arthritis and the maintaining of the adipose mass during obesity. Pathological angiogenesis is also found in psoriasis and certain forms of diabetes.

According to another advantageous embodiment of said poropeptides, they are used for countering certain other types of harmful cells, such as immune cells that recognize self and cause autoimmune diseases.

The poropeptides as defined above exhibit all the characteristics of antimicrobial peptides: amphipathic nature, cationic, destabilization of lipid membranes. The subject of the present invention is therefore also the use of the peptides as defined above for preparing a molecule with antibiotic properties capable of killing resistant bacteria.

In general, therapeutic peptides are generally metabolized and are eliminated more easily than conventional drugs, making it possible to give the patient better chances of recovery while at the same time minimizing the complications associated with standard therapies.

Furthermore, the poropeptides can be used, in accordance with the invention, as adjuvants supplementing conventional drugs, thus making it possible to reduce the doses and the side effects thereof.

According to another aspect, the invention is directed toward cosmetic compositions containing, in their active ingredient, at least one peptide as defined above, in combination with a cosmetologically acceptable vehicle.

Advantageously, the peptides of the invention exhibit stabilizing properties for these compositions.

According to another aspect, the invention is also directed toward agronomic or food-processing compositions containing, in their active ingredient, at least one peptide as defined above, in combination with a vehicle which is agronomically acceptable and acceptable in the food industry.

Other characteristics and advantages of the invention are given in the examples which follow. In these examples, reference is made to FIGS. 1 to 11 which represent, respectively,

FIG. 1, A) the “channel” domain of colicin A; B) the 3D structure of Bax; C) the α-helical hairpin motif of the proteins of the Bcl-2 family (alpha5-alpha6 in Bax);

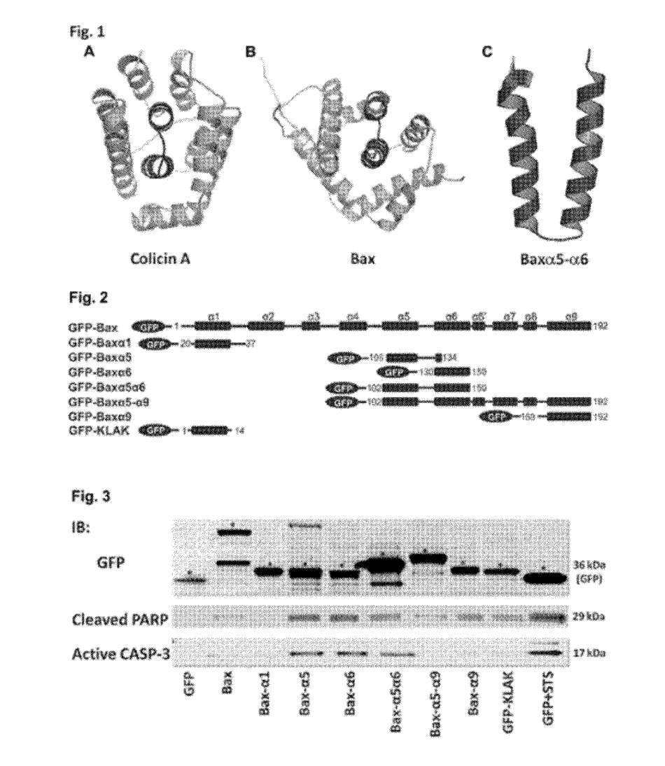

FIG. 2, constructs encoding fusions between GFP (green fluorescent protein) and the various membrane insertion or anchoring regions of the pro-apoptotic protein Bax;

FIG. 3, results of the analysis of the expression of Bax regions by Western blotting;

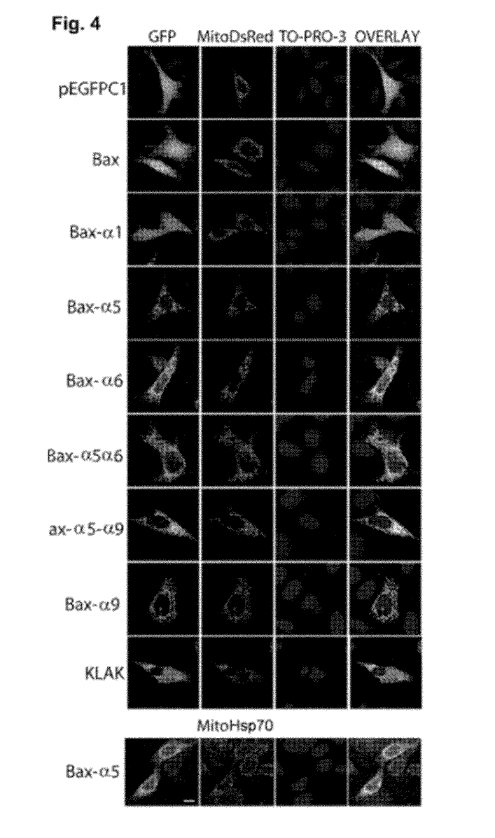

FIG. 4, cell localization of GFP with respect to said regions;

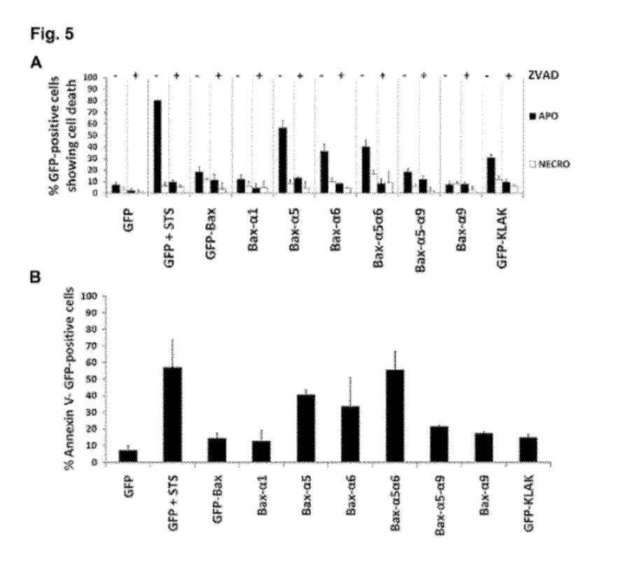

FIG. 5, death rate of cells expressing GFP or GFP fused to the peptides used according to the invention;

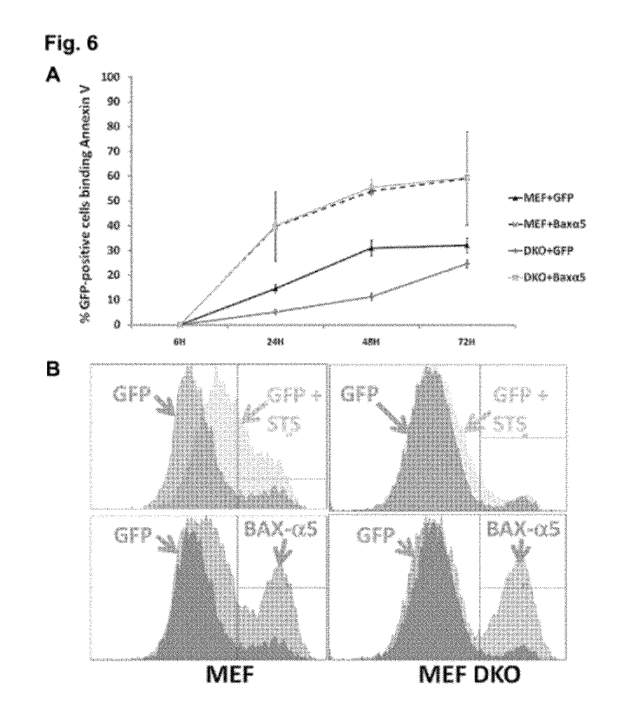

FIG. 6, level of apoptosis of wild-type murine embryonic fibroblasts (MEF) or murine embryonic fibroblasts deficient in Bax and Bak (MEF-DKO);

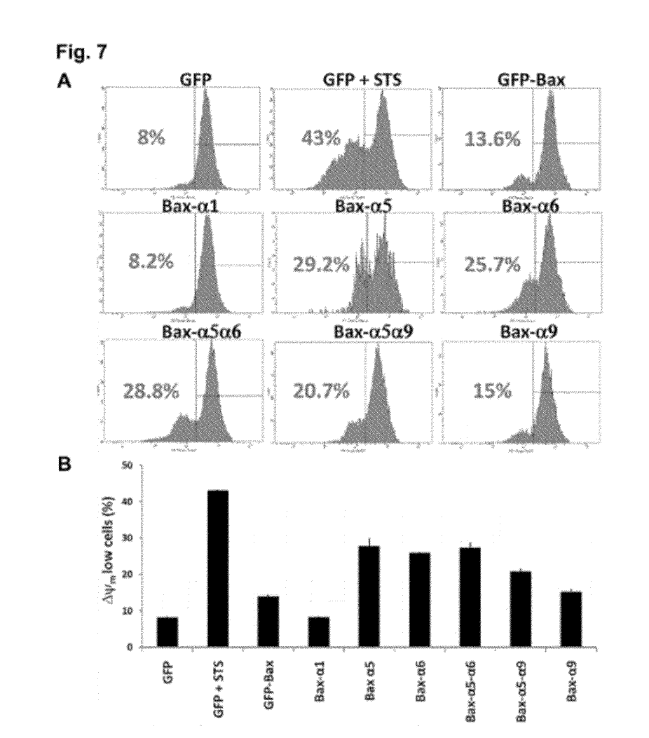

FIG. 7, mitochondrial transmembrane potential of cells expressing GFP or GFP fused to the peptides used according to the invention;

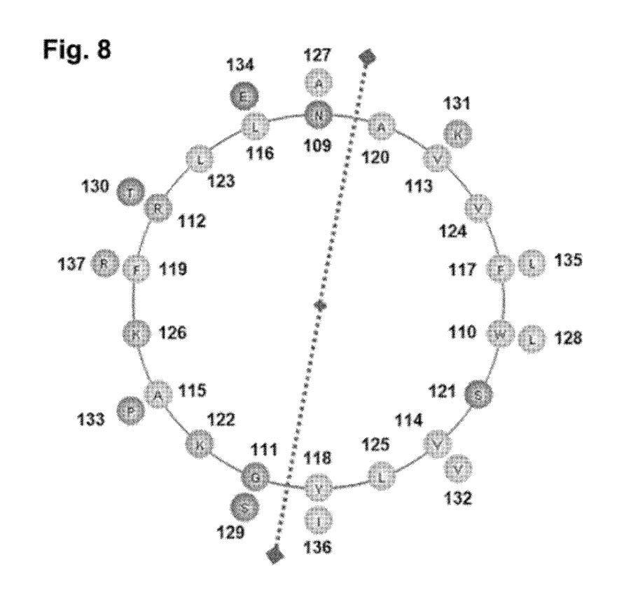

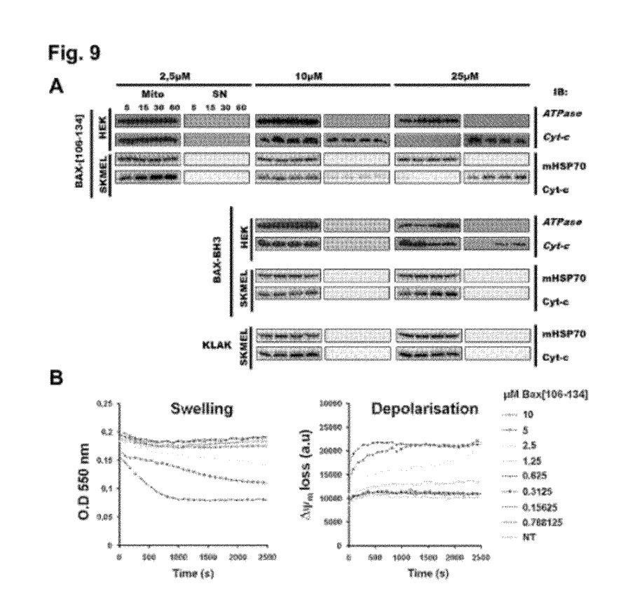

FIG. 8, the helicoidal wheel projection of a peptide of the invention;

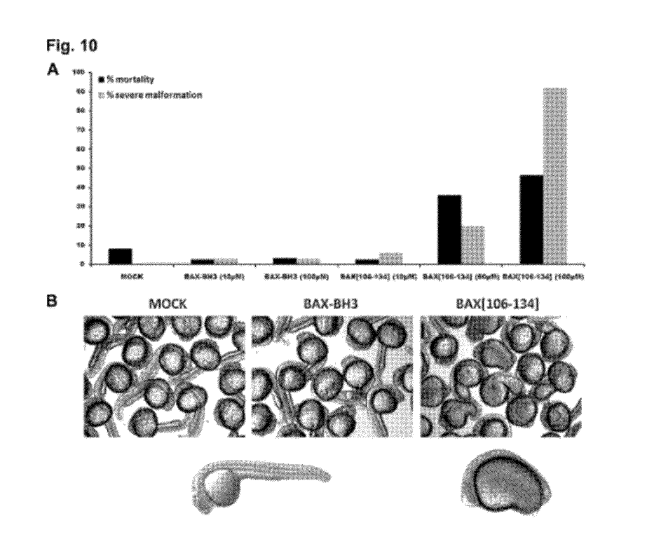

FIG. 9, the effect of a poropeptide of the invention on isolated mitochondria;

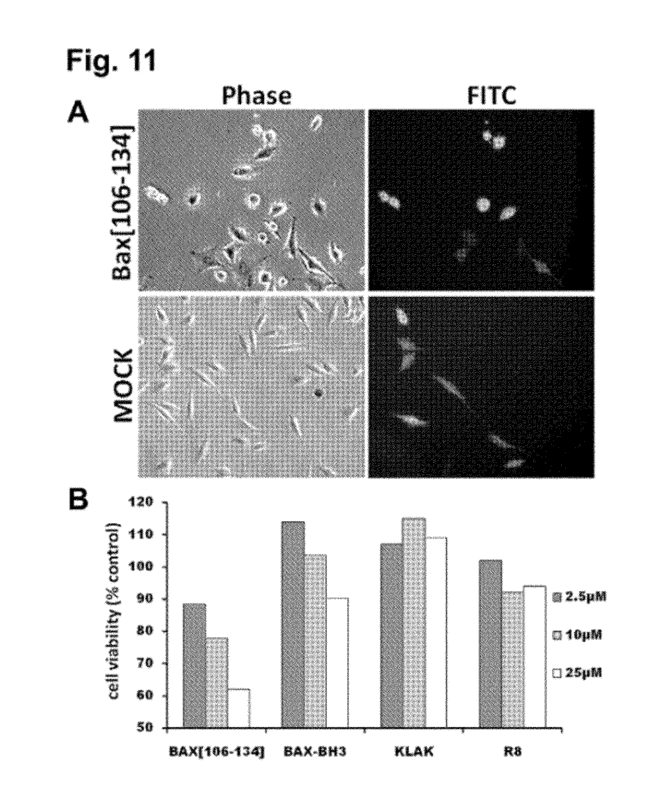

FIGS. 10 and 11, the effect of the micro-injection of peptide fragments of Bax on embryonic (FIG. 9) and cell (FIG. 10) viability and morphology; and

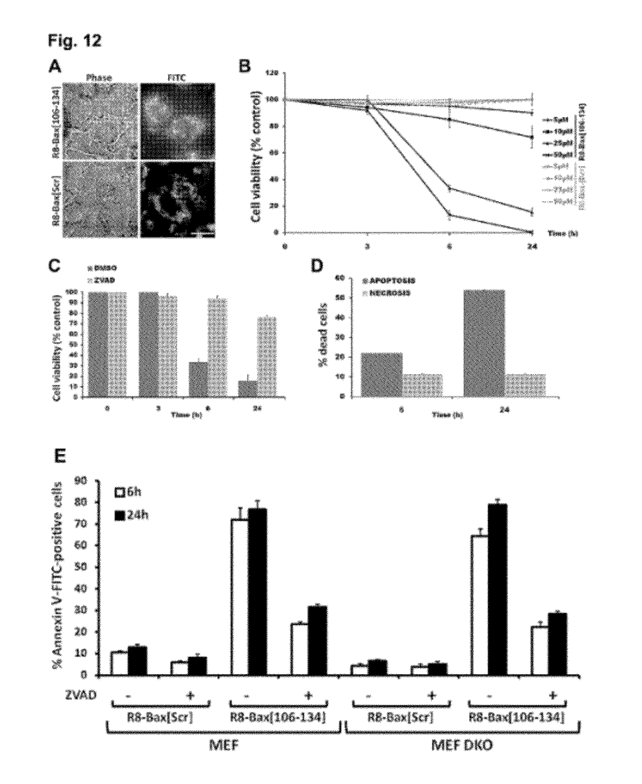

FIG. 12, the pro-apoptotic effect of a poro-peptide of the invention.

Plasmid Constructs Comprising Inserts Corresponding to the Nucleic Acids Encoding One or More Bax Regions Fused to GFP: Amino Acids 1-192 (Whole Bax Protein: α1-α2-α3-α4-α5-α6-α6′-α7-α8-α9), 20-37 (al), 106-134 (α5), 130-150 (α6), 102-150 (α5-α6), 102-192 (α5-α6-α6′-α7-α8-α9 or α5-α9), 169-192 (α9)

The nucleic acids encoding the various membrane insertion or anchoring regions present on the pro-apoptotic Bax protein were cloned into the pEGFP-C1 plasmid. The sequences inserted are given in FIG. 2. A construct (GFP-KLAK) encoding a polypeptide GFP-KLAKLAKKLAKLAK was also produced. Artificial peptides enriched in KLA sequences are known to target the negatively charged bacterial membrane and to exhibit antibiotic activity.

The recombinant plasmids were verified by molecular sequencing. The analysis of the expression of the Bax inserts, carried out by Western blotting, is reported in FIG. 3: the upper part corresponds to the analysis by Western blotting, using an anti-GFP antibody, of lysates of HT1080 fibrosarcoma cells transiently transfected with the plasmid constructs above; the middle part gives the immunoblot obtained using an antibody which recognizes the PARP apoptotic cleavage fragment; the lower part gives the immunoblot obtained using an antibody which recognizes the activated form of caspase-3.

The various constructs were transfected into human cells cultured in vitro (HT-1080 and SK-MEL-28), and then the subcellular localization and the cytotoxicity induced by the Bax membrane insertion fragments fused to GFP were determined.

FIG. 4 shows the subcellular localization of the complete GFP protein compared with that of the inserts composed of Bax membrane insertion fragments fused to GFP. MEF-DKO cells were cotransfected with the pEGFP-C1 plasmids encoding the various Bax inserts and the MitoDsRed vector. It is observed that peptides corresponding to or comprising the Bax membrane insertion helices and/or the TM domain are sufficient to target the GFP (in green) to the mitochondrial membranes (labeled red by virtue of the MitoDsRed protein, comprising the mitochondrial targeting sequence CoxVIII). Similar results were obtained using an antibody directed against the Hsp70 mitochondrial protein (insets at the bottom). These confocal microscopy results indicate that short fragments of the Bax protein (in particular the regions corresponding to amino acids 106-134, 130-150 and 169-172) contain the determinants necessary for their specific targeting to the mitochondrion.

FIG. 5 shows the cell toxicity induced by the expression of the various constructs. FIG. 5A gives the death rate (apoptosis/necrosis) of HT-1080 cells expressing GFP alone or as a fusion with various membrane insertion fragments of the Bax death protein. The cells were treated (+) or not (−) with the general caspase inhibitor zVAD.fmk (100 μM). Cell death was quantified 24 h after transfection by counting the number of green cells (expressing GFP) under an epifluorescence microscope. The type of cell death (apoptosis and necrosis) was determined through the membrane permeability to propidium iodide (necrosis) and the nuclear morphology (condensed or fragmented nuclei) after staining with Hoechst 33342 (apoptosis). The green cells negative for propidium iodide and exhibiting a pyknotic nucleus were counted as being in apoptosis. The results represent the mean values (with the standard deviations) of the three experiments.

In the test used, the green cells (expressing the construct) exhibiting a pyknotic nucleus are in apoptosis, those stained red with propidium iodide are in necrosis.

It is observed that the constructs encoding the central helices of Bax (GFP-Bax-α5α6, GFP-Bax-α5 and GFP-Bax-α6) are strongly cytotoxic and induce cell death mainly of apoptotic type. The apoptotic nature of the cell death caused by the expression of the Bax membrane insertion fragments is confirmed by the cleavage of the PARP protein and the activation of caspase-3, demonstrated by the Western blotting technique (FIG. 3) using specific antibodies, and by blocking of the apoptosis with a broad-spectrum caspase inhibitor (zVAD.fmk) (FIG. 5A). In addition, the triggering of apoptotic cell death is demonstrated by labeling with Annexin-V, which specifically recognizes phosphatidyl-serines externalized by cells in apoptosis (FIG. 5B). In this experiment, the cell death was quantified using the Annexin-V-Cyanine 3 test by flow cytometry 24 h after transient transfection of the cells with the plasmids of interest. It should be noted that the GFP-Bax-α5 and GFP-Bax-α6 constructs are more effective for inducing apoptosis than the GFP-KLAK construct.

FIG. 6 shows that the toxic proteins encoded by the inserts do not act by means of the endogenous Bax and Bak pro-apoptotic proteins, since murine fibroblasts deficient in Bax and Bak (MEF-DKO) are not resistant to stress induced by their expression (see also FIG. 3). The graph (FIG. 6A) corresponds to a time course of cell death (quantified by flow cytometry by means of the Annexin V-Cy3 test) of MEF wild-type murine fibro-blasts compared with MEF-DKO murine fibroblasts. It is observed that the Bax- and Bak-deficient fibroblasts are as sensitive to the induction of apoptosis through the expression of the GFP-Bax-alpha5 protein as wild-type murine fibroblasts. At the same time, the MEF-DKO cells are resistant to apoptosis induced by staurosporine (FIG. 6B). These results indicate that the Bax-alpha5 region is capable of killing MEF-DKO cells by apoptosis, said cells being described as resistant to a large number of apoptotic stimuli (Wei et al., Science, 2001).

FIG. 7 shows that cells (HT-1080) expressing the polypeptides comprising the α5 and/or α6 helices of Bax experience a considerable drop in their mitochondrial transmembrane potential (Δψm), contrary to the cells expressing GFP alone, suggesting the involvement of a mitochondrion-dependent pathway in the induction of apoptosis. The cells were stained with Mitotracker 24 h after transfection and analyzed by flow cytometry (FIG. 7A). The histograms (FIG. 7B) show the mean values of three experiments of flow cytometry quantification of the green cells (expressing GFP) exhibiting Mitotracker incorporation deficiencies (i.e. a low Δψm).

The results presented show that the regions of the Bax pro-apoptotic protein corresponding to amino acids 106-104 and 130-150 are capable of targeting mitochondrial membranes and of inducing cell suicide by apoptosis. These regions are structured as alpha-helices within the complete Bax protein (Suzuki et al., 2000, Cell, 103, 645-654) and have an amphiphatic nature favorable to the destabilization of biological membranes.

### Poropeptide of Sequence SEQ ID No. 1

It is an amphiphatic peptide (see FIG. 8), longer than the α5 helix deduced from the 3D structure of the Bax protein in solution (Suzuki et al., 2000). It comprises a serine residue incorporated in position 126 instead of the natural cysteine residue, in order to limit the disulfide bridge formation.

Characterization of its Mode of Action by Incubation with Isolated Mitochondria

The ex cellulo activity of poropeptide-Bax (106-134) was tested by determining its ability to promote the release of cytochrome c from the intermembrane space of isolated mitochondria. The results obtained are shown by FIG. 9 (A).

The peptide is incubated with the mitochondria for 5, 15, 30 or 60 min, and the mitochondrial release of cytochrome c into the supernatant (SN) is measured by Western blotting. The mitochondrial fractions (Mito) are calibrated using an antibody directed against the HSP70 or ATPase (subunit 6) mitochondrial proteins. With poropeptide-Bax (106-134), cytochrome c is released from the intermembrane space of the mitochondrion at 10 μM and in 5 min of incubation. No release is observed when an artificial antimicrobial peptide (KLAK) of sequence SEQ ID No. 2 KLAKLAKKLAKLAK is used.

At 10 μM, which corresponds to the minimum concentration for which release is observed with poropeptide-Bax (106-134), no release is observed for the Bax-BH3 peptide (BH3 death domain of Bax corresponding to the α2 helix, located about thirty residues upstream of the α5 helix). Cytochrome c is released from the mitochondrial intermembrane space starting from 25 μM using mitochondria isolated from HEK293T cells and is never released with SK-MEL-28 cells. Poropeptide-Bax (106-134) is therefore more active than Bax-BH3 for releasing cytochrome c from the intermembrane space of the mitochondrion. Furthermore, it is more effective, since it allows release as early as the first 5 minutes, whereas it is necessary to wait 30 min for the BH3 peptide in the HEK293T cells.

These experiments show that poropeptide-Bax [106-134], of sequence SEQ ID No. 1, has a strong capacity for inducing mitochondrial membrane permeabilization.

FIG. 9B shows, in addition, that poropeptide-Bax (106-134) profoundly impairs the physiology of the organelle, by causing a physical process of swelling (left-hand panel) and of depolarization (right-hand panel). Poropeptide-Bax (106-134) induces swelling of the mitochondria (DD₅₀=1.68±0.39 μM) and a drop in their transmembrane potential (SD₅₀=3.98±0.57 μM). These two parameters were measured using mitochondria isolated from rat liver, incubated in the presence of various concentrations of poropeptide-Bax (106-134) (NT=not treated).

These results obtained in vitro reinforce the involvement of a mitochondrion-dependent pathway in the induction of apoptosis by the α5 region of Bax.

Induced-Cytotoxicity Measurements

Endogenous Administration of the Peptide (by Microinjection)

Poropeptide-Bax [106-134] was microinjected in vivo. The zebrafish (Danio rerio) model was used because (i) the apoptotic machinery is conserved between human cells and fish cells; (ii) the egg corresponds to an integrated cell system compared with isolated mitochondria; (iii) zebrafish eggs can be easily injected routinely.

The results obtained are shown in FIG. 10:

(A) Zebrafish eggs were microinjected at the 1-2 cell(s) stage with increasing concentrations of poro-peptide-Bax [106-134], of Bax-BH3 peptide or with ultrapure water ('mock'). A concentration of 100 μM inside the microinjection capillary corresponds to approximately 6×10¹² molecules of peptide per egg. The histograms represent the percentage mortality 24 h after fertilization and the percentage of surviving embryos exhibiting severe malformations. The data shown are representative of 2 independent experiments giving the same results.
(B) Embryonic morphology 24 h after microinjection (100 μM of peptide). Severe malformations can be observed in the group of fish injected with poropeptide-Bax [106-134] compared with the group injected with the BH3 peptide.

Poropeptide-Bax [106-134] was then mixed with a solution of dextran-fluorescein (so as to allow its visualization by epifluorescence microscopy), and then microinjected into SK-MEL-28 human melanoma cells. Cell viability was then monitored and quantified 12 hours after microinjection.

FIG. 11 gives the results obtained:

(A) Micrographs showing SK-MEL-28 human melanoma cells injected with Dextran-FITC fluorescent tracer alone (‘mock’) or mixed with poropeptide-Bax [106-134]. The cells were photographed 12 h after microinjection. The cells microinjected with poropeptide-Bax [106-134] exhibit characteristic morphological manifestations of apoptosis (cytoplasmic budding, shrinkage and cell rounding). Magnification×800.
(B) Cell viability after microinjection of poropeptide-Bax [106-134] or of Bax-BH3, KLAK or R8 peptide. The cells microinjected were visualized by means of the Dextran-FITC tracer. Cell death was estimated 12 h after microinjection by counting the number of peptide-microinjected cells exhibiting typical morphological manifestations of apoptosis, compared with control (microinjection of ultrapure water supplemented with dextran-fluorescein). The data shown are representative of 2 independent experiments giving the same results.

The results show that microinjection of poropeptide-Bax [106-134] induces substantial cell death, whereas the cells microinjected with Bax-BH3 or KLAK have no significant effect. The cells microinjected with control peptide (R8) of sequence SEQ ID No. 3 RRRRRRRR (a polyarginine intracellular transduction motif) exhibit viability rates comparable to the control. These data show that poropeptide-Bax [106-134] exerts dose-dependent cytotoxic effects after introduction into zebrafish eggs or into tumor cells cultured in vitro, and that it is more effective for inducing cell death than a pro-apoptotic peptide resulting from the same protein (Bax-BH3) or than other peptides known to be active at the membrane level (KLAK and R8).

Exogenous Administration (by Coupling with a Transducer Peptide of Polyarginine Type)

The poropeptide of sequence SEQ ID No. 1, comprising a polyarginine N-terminal extension (R8) separated by a glycine residue, was used. A fluorescein isothiocyanate (FITC) group was added in the C-terminal position so as to allow its visualization by epifluorescence microscopy. The R8-Scr-FITC peptide is a “scramble” sequence of the sequence present in the R8-Bax [106-134]-FITC peptide, i.e. a control sequence in which the amino acids (their order in the sequence) have been mixed-up.

FIG. 12 shows the results obtained:

(A) HeLa cells were incubated for 6 h in the presence of 10 μM of poropeptide FITC-R8-Bax [106-134] or of control peptide R8-Bax [Scr] (“scramble” version of the poropeptide), and then observed under an epifluorescence microscope. The cells incubated in the presence of the fluorescent peptides exhibit strong cytoplasmic labeling (visible from 1 h of incubation onward). Scale 10 μm.
(B) Poropeptide R8-Bax [106-134] induces a loss of cell viability which is dose-dependent and incubation time-dependent. HeLa cells were treated with increasing concentrations (5, 10, 25 and 50 μM) of peptide R8-Bax [106-134] or R8-Bax [106-134]. The cytotoxicity was determined by measuring the cellular release of lactate dehydrogenase (LDH) after 3, 6 or 24 h of incubation (n=4). The peptide R8-Bax [Scr] does not induce any significant leaking of LDH at the concentrations tested. The results are given in means and standard deviations.
(C) The general caspase inhibitor zVAD.fmk reduces cell death induced by poropeptide R8-Bax [106-134] (25 μM). The cytotoxicity was determined by measuring the cellular release of lactate dehydrogenase (LDH) after 3, 6 or 24 h of incubation in the presence of 100 μM of zVAD.fmk or of buffer alone (‘DMSO’). The results are given as means and standard deviations.
(D) Death rate (apoptosis/necrosis) of HeLa cells treated with poropeptide R8-Bax [106-134]. The type of cell death was determined 24 h after transfection by observation of cell permeability to propidium iodide (necrosis) or of nuclear morphology after DNA staining with Hoechst 33342 (apoptosis). The green cells (expressing GFP) were visualized by epifluorescence microscopy. Approximately 300 cells were counted per experiment. The results are a mean of three independent experiments (the standard deviation is also represented).

Thus, the two peptides (poropeptide FITC-R8-Bax [106-134] and “scramble” control peptide R8-Bax [Scr]) are rapidly internalized (in less than one hour) by HeLa cells in culture in vitro (FIG. 12A). Unlike the control peptide, poropeptide R8-Bax [106-134]-FITC inhibits HeLa cell viability in a dose-dependent and incubation time-dependent manner (FIG. 12B). A pan-caspase inhibitor, zVAD.fmk, added in vitro, significantly inhibits the cell death induced by R8-Bax [106-134]-FITC (FIG. 12C), indicating the participation of caspases in its pro-apoptotic activity (FIG. 12D).

(E) Level of apoptosis of wild-type murine fibroblast cells (MEF) or murine fibroblast cells deficient in Bax and Bak death proteins (MEF DKO), treated with poropeptide FITC-R8-Bax [106-134] or the control peptide FITC-R8-Bax [Scr]. The apoptosis was quantified using the Annexin V-Cyanine 3 test by flow cytometry 6 h or 24 h after treatment, in the presence or absence of the general caspase inhibitor zVAD.fmk. The results correspond to the percentage of apoptotic cells having internalized the fluorescent peptide under each condition.

1. The use of derivatives of the membrane insertion helices of the proteins of the Bcl-2 family for inducing cell death by targeting the mitochondrion, characterized in that the derivatives are peptides corresponding to the α5 and/or α6 regions of the Bax protein or to the equivalent regions present in the other proteins of the Bcl-2 family.

2. The use as claimed in claim 1, characterized in that said peptides are developed from the regions corresponding to the amino acids in position 106-134, 130-150 and 102-150 of the Bax protein.

3. Peptides derived from the α5 and/or α6 regions of the Bax protein or from the equivalent regions present in other proteins of the Bcl-2 family.

4. The peptides as claimed in claim 5, characterized in that they comprise means for cell penetration or recognition of a particular cell type.

5. The peptides as claimed in claim 6, characterized in that said means correspond to a transduction domain such as a polyarginine motif.

6. The peptides as claimed in claim 5, characterized in that one or more amino acids are deleted or substituted with another.

7. The peptides as claimed in claim 8, characterized in that one or more cysteine residues are replaced with serine or glycine residues.

8. A peptide of sequence SEQ ID No. 1: NH₂-NWGRVVALFYFASKLVLKALSTKVPELIR-COOH.

9. Pharmaceutical compositions, characterized in that they contain a therapeutically effective amount of at least one peptide as defined in claim 3, in combination with a pharmaceutically acceptable vehicle.

10. Cosmetic compositions, characterized in that they contain, in their active ingredient, at least one peptide as defined in claim 3, in combination with a cosmetologically acceptable vehicle.

11. Food-processing or agronomic compositions comprising, in their active ingredient, at least one peptide as defined in claim 3, in combination with a vehicle which is agronomically acceptable and acceptable in the food industry
